(19) United States
(12) Patent Application Publication
(10) Pub. No.: US 2005/0106566 A1
(43) Pub. Date: May 19, 2005

(54) Substrate coated with a transparent organic film and manufacturing process

(76) Inventors: Francois Breniaux, Massy (FR); Christophe Bureau, Suresnes (FR); Patrick Chaton, Theys (FR); Stephane Getin, Grenoble (FR)

Correspondence Address: BURNS DOANE SWECKER & MATHIS L L P, POST OFFICE BOX 1404, ALEXANDRIA, VA, 22313-1404, US

(21) Appl. No.: 10/493,041

(22) PCT Filed: Oct. 16, 2002
(86) PCT No.: PCT/FR02/03538

(30) Foreign Application Priority Data
Oct. 18, 2001 (FR) 01/13432

(51) Int. Cl. H01L 21/00; C12Q 1/68; C12M 1/34

(52) U.S. Cl. 435/6; 438/1; 438/780; 435/287.2

(57) ABSTRACT

The present invention relates to a substrate coated with a transparent organic film, to a process for the manufacture of this substrate coated with the transparent organic film and to its use. The substrate coated with a film is characterized in that the film is an electrical insulator organic polymer which is transparent in at least one wavelength range and in that the said film is combined with a label which emits at least in the said wavelength range. It has an application in particular in a means for the detection of a chemical entity, for example a biochip, in a process for the quality control, a process for the certification or a process for the authentication of an object.

### TECHNICAL FIELD

The present invention relates to a substrate coated with a transparent organic film, to a process for the manufacture of this substrate coated with a transparent organic film, and to its use.

The term “transparent film” is understood to mean a film having noteworthy optical properties of transparency (absence of optical absorption, absence of “quenching”, and the like) over at least one region of the electromagnetic spectrum and in particular a low optical extinction or “coefficient k” within the wavelength region under consideration for the transparency. This optical extinction coefficient is measured, for example, by spectroscopic ellipsometry or using spectrophotometry.

The substrate can be an insulating, conducting or semiconducting substrate with regard to electricity. It constitutes the support for the transparent organic film and it is chosen according to the use or the destination of the substrate coated with the transparent organic film in accordance with the present invention.

The present invention has an application, for example, in the field of the quality control, of the certification or of the authentication of substrates coated with thin transparent films.

This is because it makes it possible to prove the manufacture and/or the origin of the substrate coated with the organic film of the present invention, in or on which it will have been possible to deliberately insert a known fluorescent, phosphorescent or chemiluminescent label in very small amounts. In this application of the present invention, the substrate is any object.

It also has an application, for example, in the field of the detection of the chemisorption or physisorption, on or in the transparent organic film, of chemical, biochemical or biological entities functionalized beforehand by a fluorescent, phosphorescent or chemiluminescent label, such as, for example, in processes for the detection by fluorescence of chemical or biochemical analysis chips, such as DNA chips. In this type of application, the substrate constitutes the support of the detection means.

For example, in the case of a biochip, the substrate can, for example, be a support formed of silica, of gold or of a composite, such as Au/Si, Au/SiO₂ or more generally metal/substrate, and the transparent organic film can be one of the molecular means for attaching biological probes to certain parts of the surface.

When the biochip is brought into contact with a solution of sample to be analyzed, pairings take place between the DNAs of the sample and those attached to the substrate. This attachment can, for example, be detected by having labelled the DNAs of the sample beforehand with a fluorescent, phosphorescent or chemiluminescent label. In accordance with the present invention, the film is chosen in order to be transparent at the emission wavelength of the fluorescent, phosphorescent or chemiluminescent label used, so as to absorb as few as possible of the photons emitted by this label, to render it detectable at very low concentrations at the surface of the substrate and to minimize the interference with the measurement. The signal/noise ratio and the lower detection limits of the pairings on the biochips are thus found to be improved thereby.

In the description which follows, the references in square brackets refer to the appended list of references.

### PRIOR ART

As regards the biochip application, the documents FR-A-2 787 581 (1998), FR-A-2 787 582 (1998) and U.S. Pat. No. 5,810,989 (1998) disclose the electrocopolymerization on a silica substrate of precursor monomers of conducting polymers, such as pyrrole, with monomers functionalized by recognition molecules, in particular oligonucleotides. This technique is among the currently most widely used techniques for the localized attachment of recognition molecules to the plots of a biochip.

In this technique, use is made of the adhesion of the conducting polypyrrole film to the substrate, so as to carry out the attachment thereto of the recognition molecules.

As disclosed in the abovementioned documents, and in the documents FR-A-2 784 188 (1998) and FR-A-2 784 189 (1998), the chip thus functionalized is brought into contact with a solution of sample to be analyzed comprising target molecules capable of coupling with the recognition molecules of the support.

In order to selectively detect the plots on which a coupling is present between the recognition molecules and the target molecules, the latter can advantageously be “labelled” with a fluorescent molecule, such as, for example, fluorescein or phycoerythrin, which exhibits an absorption at 543 nm and an emission at 580 nm, the presence of which can subsequently be detected using an appropriate optical device.

Unfortunately, polypyrrole has a not insignificant absorption in the emission wavelength region of the fluorescent label used. This disadvantage is widely described in the literature relating to the techniques concerned.

Specifically, Arwin et al., in the reference [1], measure an extinction coefficient k=0.3, for a refractive index n=1.45, on a polypyrrole film with a thickness of 22 nm on gold; Kim et al., in the reference [2], measure an extinction k=0.3 and an index n=1.6 at λ=632.8 nm, for a polypyrrole film with a thickness of 47 nm; Kim et al., in the reference [3], measure an index n=1.45 for an extinction k=0.28 on a polypyrrole film with a thickness of 54 nm in the oxidized state and an index n=1.6 for an extinction k=0.21 on a film with a thickness of 47 nm in the reduced state; Guedon et al., in the reference [4], measure an index n=1.7 for an extinction k=0.3 at 633 nm on a polypyrrole film on gold and for thicknesses of the film of between 7.5 and 20 nm.

Consequently, the polypyrrole used to attach the recognition molecules absorbs a large part of the fluorescence signal of the coupled target molecules. It thus interferes with the detection method.

In addition, as the target molecules and their labels are included in the polypyrrole, in particular owing to the fact that the recognition molecules have been attached by copolymerisation, this interfering absorption increases the value of the lower detection limit which can be achieved by this process.

In the field of diagnosis, for example, this disadvantage is a great nuisance, since it is, specifically at low concentrations of target molecules, thus at low surface concentrations of fluorescent labels, that all the advantages of the biochip lie.

### ACCOUNT OF THE INVENTION

An aim of the present invention is in particular to provide a substrate coated with a thin transparent film and a process for the manufacture of this substrate coated with the film which, first, responds to the technical problems posed in the prior art relating to biochips and, secondly, provides a novel powerful tool in the fields of the quality control, of the certification and of the authentication of any objects.

The substrate coated with a film of the present invention is characterized in that the film is an electrical insulator organic polymer which is transparent in at least one wavelength range and in that the said film is combined with a label which emits at least in the said wavelength range.

This is because the inventors have demonstrated that, unexpectedly, the electrical conductor nature and the high optical extinction are connected.

Thus, according to the present invention, the term “transparent” is intended to mean transparent at the detection wavelength of the label used in accordance with the present invention.

According to a first embodiment of the present invention, the wavelength range in which the insulating polymer has to be transparent according to the present invention is determined according to the label used, for example, in the abovementioned applications.

In particular, once the label, and thus the emission wavelength of the label, has been chosen, it is easy to determine the value of the extinction of such and such a polymer at the desired wavelength, for example by spectroscopic ellipsometry or using spectrophotometry, and to examine whether it can be used according to the invention.

According to this first embodiment of the present invention, it is thus the insulating polymer which is chosen as a function of the label.

According to a second embodiment of the present invention, after having chosen an insulating polymer, the wavelength range within which it is transparent is determined and then, according to this wavelength range, a label which emits in the said transparency range of the polymer is selected.

According to this second embodiment of the present invention, it is thus the label which is chosen as a function of the polymer.

According to the present invention, any insulating polymer is therefore capable of being used as a transparent film over at least one wavelength range.

Mention may be made, among these, as nonlimiting examples, of vinyl polymers, vinyl copolymers and their blends, which may or may not be crosslinked, and in particular of polymers, copolymers and their blends, which may or may not be crosslinked, of acrylonitrile, of methacrylonitrile, of methyl methacrylate, of ethyl methacrylate, of propyl methacrylate, of butyl methacrylate, of hydroxyethyl methacrylate, of hydroxypropyl methacrylate, of cyano acrylate, of acrylic acid, of methacrylic acid, of styrene, of para-chlorostyrene, of N-vinylpyrrolidone, of vinyl halides, of acryloyl chloride or of methacryloyl chloride.

Mention may also be made, as nonlimiting examples, of the crosslinked or non-crosslinked polymers chosen from polyacrylamides, polymers of isoprene, of ethylene, of propylene, of ethylene oxide and molecules comprising strained rings, of lactic acid or of its oligomers, of lactones, of ε-caprolactone or of glycolic acid, aspartic acid, polyamides, polyurethanes, parylene and polymers based on substituted parylene, oligopeptides and proteins, and the prepolymers, macromers or telechelics based on these polymers, and the copolymers and/or blends which can be formed from the monomers of these polymers or from these polymers themselves.

The choice of the insulating polymer to be used for the application under consideration, for example among the abovementioned insulating polymers, can subsequently be determined by considerations other than those strictly related to the optical properties of the material.

Thus, according to the invention, the polymer can be selected, within the range of the polymers which can be used according to the invention, for example from a polymer capable of adhering to a substrate, a polymer capable of being functionalized, a polymer having thermoelastic properties, and the like.

Among the abovementioned insulating polymers, the inventors have directed their attention more specifically at vinyl polymers because they can be easily manufactured by various types of reactions, for example ionic or radical reactions, in particular as thin films, and because they can also be obtained by the electrochemical route and can be electrografted to surfaces which are conducting or semiconducting with regard to electricity.

The vinyl polymers are obtained by polymerization of monomers in following generic formula (I): 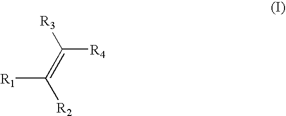
in which R₁, R₂, R₃ and R₄ are hydrogen atoms or organic groups of any nature, for example from hydrocarbons chosen, for example, from alkanes, alkenes or alkynes; for example amides, aldehydes, ketones, carboxylic acids, esters, acid halides, anhydrides, nitriles, amines, thiols, phosphates, ethers, homo- or heterocyclic aromatics or any cyclic group comprising these functional groups, and any group carrying several of these functional groups;
or by polymerization of a mixture of different monomers corresponding to the formula (I) above.

In the nonlimiting examples of the implementation of the present invention set out below, the optical properties of polymethacrylonitrile (PMAN), with R₁=R₂=H, R₃=CH₃ and R₄=CN, and of poly(methyl methacrylate) (PMMA), with R₁=R₂=H, R₃=CH₃ and R₄=C(═O)OCH₃, were examined. These two monomers can result in insulating polymers electrografted to conducting surfaces by electroreduction in an aprotic organic medium.

According to the invention, in a specific application, the vinyl polymers can be grafted, optionally in a selective and localized way, to the conducting or semiconducting surfaces of a substrate by electrografting vinyl monomers, which renders them advantageous as substitutes for polypyrroles in a biochip application.

These polymers can be deposited on surfaces of any type following the application of the present invention by the various processes known to the person skilled in the art for depositing thin polymer films, such as the techniques of spin coating; of dipping; of vaporization under ultra-high vacuum; of CVD; of surface chemical polymerization, as disclosed, for example, in U.S. Pat. No. 4,421,569, U.S. Pat. No. 5,043,226 or U.S. Pat. No. 5,785,791; of photochemical grafting of polymers to surfaces, as disclosed, for example, in Patent Applications WO-A-9908717 and WO-A-9916907; of grafting of polymers under irradiation of particles or photons; of chemical grafting to a surface of oxide or of another polymer, either directly or via chemical coupling agents (such as thiols, silanes, and the like); of depositions following a polymerization brought about by initiators, in particular radical initiators, which depositions are obtained in situ by electrochemistry; and the like.

According to a specific embodiment of the invention, it is possible, for example, to obtain a deposit of transparent polymer as a thin layer by polarizing a conducting or semiconducting surface in a solution or in a gel comprising in particular diazonium salts and monomers which can be polymerized by the radical route.

According to an advantageous embodiment, such polymer films are grafted to the surface of the substrate in particular as thin films, that is to say with thicknesses of less than one micrometer, for example of between 1 and 100 nm.

Other thicknesses are possible in the context of the present invention as defined in the appended claims.

The films can be either preformed polymer films grafted to the surface, for example by the chemical, electrochemical or photochemical route, in one or more stages, or films constructed directly on the surface from precursor monomers, for example initiated by the chemical, electrochemical or photochemical route.

This grafting can be carried out, according to the physicochemical characteristics of the process employing the present invention, on surfaces which are insulating, conducting or semiconducting with regard to electricity.

According to a favoured embodiment, ultrathin films of transparent polymers can be obtained on surfaces which are conducting or semiconducting with regard to electricity by electrografting vinyl monomers, for example as disclosed in Patent Application EP-A-038 244.

According to the present invention, the label can be any label provided that it can be combined with an electrical insulator organic polymer in accordance with the present invention, the said polymer having to be transparent in at least the emission wavelength range of the label.

For example, the label can be a fluorescent, phosphorescent or chemiluminescent label.

It can be, for example, a label chosen from fluorescein or substituted fluoresceins, such as fluoresceindiacetate, 5- and 6-carboxyfluoresceins, 5- and 6-carboxyfluoresceindiacetates, the succinimidyl ester of 5- and 6-carboxyfluoresceins, and the like; rhodamine and substituted rhodamines; coelenterazine and substituted coelenterazines; aequorin; luciferin and substituted luciferins; bromochloroindoxyl phosphates (BCIP); luminol; nonyl acridine orange (NAO); 5, 5′, 6, 6′-tetrachloro-1, 1′, 3, 3′-tetraethylbenzimidazolyl-carbocyanine chloride; 4-(4-ditetradecylaminostyryl)-N-methylpyridinium iodide; 1,1′-dioctadecyl-3,3,3′,3′-tetramethylindocarbocyanine perchlorate; 3,3′-dihexadecyloxacarbocyanine hydroxyethanesulfonate; bis-(1,3-dibarbituric acid)-trimethine oxanol; tetrazolium salts; calcium complexes; potassium complexes; anthraquinone; anthracene; pyrene; doxorubicin; phycoerythrin; porphyrins; phthalocyanines and more generally organometallic complexes; fluorescent proteins and in particular GFPs, Green Fluorescent Proteins (K. F. Sullivan, S. A. Kay, “Methods in Cell Biology: Volume 58: Green Fluorescent Proteins”, Academic Press, 1999); salts of fluorescent minerals (and in particular uranium salts); and any molecule having a fluorophore group. A chemiluminescent label is a label which emits a fluorescence when it is brought into the presence of another molecule. These labels are known to a person skilled in the art; it can be, for example, the biotin-avidin pair commonly used in molecular biology.

The amount of labels can be very low in view of the choice of the combined electrical insulator organic polymer. For example, the label can be at a concentration of the order of the nanomolar to the micromolar, for example from 1 nM to 10 μM.

According to the invention, the term “combined label” is understood to mean a label mixed with the insulating organic polymer film, or attached to the monomer(s), for example functionalized beforehand, used for the manufacture of the polymer film, or attached directly or indirectly to the surface of the film, or trapped in the polymer film during the manufacture of the latter on the surface, or simply deposited on the film using a solution of the label.

For example, in the quality control, certification or authentication applications, the label can be mixed with the insulating organic polymer, or inserted after deposition of the polymer by dipping in a solution of a solvent which swells the polymer comprising the label, or inserted during the synthesis by carrying out the polymerization in the presence of the label in the synthesis medium, or inserted during the synthesis by carrying out the polymerization with monomers or comonomers chemically functionalized with the label, or deposited on the polymer using a solution of labels.

For example, in the applications relating to biochips, the label can be combined by directly grafting onto the film or by indirectly grafting onto recognition molecules grafted to the insulating organic polymer film.

Thus, the present invention makes it possible, for example, to solve the problems of the abovementioned prior art relating to biochips by substituting the commonly used polypyrrole by an insulating polymer in accordance with the present invention, for example by a vinyl polymer. This is because, as demonstrated below in the implementational examples of the present invention, these insulating polymers have extinctions which are 10 to 100 times smaller than those of conducting polymers such as polypyrrole, which greatly reduces their interference with a label in detection processes using a chip, for example a biochip.

Consequently, the present invention makes it possible to manufacture detection chips which are much more sensitive than those of the prior art by lowering the lower detection limit, for example with respect to chips using polypyrrole or another conducting polymer.

These detection chips can be manufactured by any known means, except that the conducting films deposited on the substrates will have to be replaced by insulating films chosen in accordance with the present invention.

The present invention also relates to the use of such thin transparent organic films.

Generally, such a coating is capable of accommodating a label, for example a fluorescent, phosphorescent or chemiluminescent label, chosen within the region of the electromagnetic spectrum corresponding to its area of transparency and of allowing the detection of this label even, and in particular, when this label is present at very low concentrations in or on the organic film, this being the situation by virtue of the optical transparency properties of the insulating organic polymer film chosen.

Thus, the present invention relates to the use of a film of electrical insulator organic polymer which is transparent in at least one wavelength range combined with a label which emits at least in the said wavelength range in a process for the detection of a chemical entity.

This is because the film of insulating organic polymer of the present invention can be functionalized with recognition molecules for the chemical entity, such as nucleic acids, proteins, antigens, antibodies, synthetic organic molecules, and the like. For example, in a process for detection by biochip, the chemical entity can be DNA.

The film of insulating organic polymer of the present invention can also be used to encapsulate molecules, for example bioactive molecules, such as doxorubicin, the molecular structure of which is such that these molecules are fluorescent. In this application, the molecule has two properties belonging to it alone: that related to its bioactivity and that related to its fluorescent nature.

More generally, the present invention relates to the use of the film of electrical insulator organic polymer of the present invention which is transparent in at least one wavelength range combined with a label which emits at least in the said wavelength range in a means for the detection of a chemical entity.

As set out above, the detection means can be a biochip, such as a DNA chip, a protein chip, a chemical probe, and the like.

The present invention also relates to the use of the film of electrical insulator organic polymer of the present invention which is transparent in at least one wavelength range combined with a label which emits in the said wavelength range in a process chosen from a process for the quality control, a process for the certification or a process for the authentication of an object.

In the quality control of an industrial process for the deposition of thin insulating polymer films, for example, it is essential to be able to characterize the thickness of the coating. On thin films, in particular when these films have a thickness of less than 100 nm, it is necessary to resort to expensive and slow techniques, such as profilometry or ellipsometry, to obtain reliable measurements of the thicknesses. In addition, rapid measuring devices often have a lower detection limit of greater than one micron. Furthermore, when the samples to be described are complex in shape (microbeads, meshes, powders, and the like), the measurements, for example by profilometry or ellipsometry, are difficult.

By combining, according to the present invention, a polymer film and a label, for example a fluorescent, phosphorescent or chemiluminescent label, it is possible to obtain, in a very simple way, an indirect measurement of the thickness of the film by measuring the intensity of the fluorescence emitted.

This can be carried out, for example, by subjecting the object on which the polymer film combined with the label according to the present invention has been deposited to irradiation by a light source, the spectrum of which contains at least the absorption wavelength of the fluorophore, and by measuring the intensity of the fluorescence emitted over the emission wavelength of the fluorophore.

For this, it is sufficient to prepare beforehand a calibration curve, or standard curve, on which the intensity of fluorescence of a film combined with a fluorescent label is plotted as a function of the thickness of the film, measured by profilometry or ellipsometry, for various flat samples covered with the polymer film according to the industrial deposition process in question, the label being combined with the film at a chosen concentration identical for all samples.

From this standard curve, the measurement of the intensity of fluorescence emitted by an identical polymer film manufactured by the industrial process in question and comprising the chosen concentration, which is identical to that of the abovementioned samples, of label is sufficient to determine whether the film exhibits the required thickness, that is to say whether it does or does not meet the specifications in terms of thickness, from the knowledge of the area of the said object.

If this area is not known, the same protocol provides at least a means for the in-line monitoring of the reproducibility of the industrial method for the deposition of a polymer film.

The monitoring of the thickness of the films according to the present invention, must be carried out in a very short time from a standard curve.

In this quality control application, the present invention makes use of the active properties of the coating because of the involvement of a fluorophore.

The present invention can also be used in the certification or the authentication of an object. For this, it is sufficient to deposit, on the said object, a film of an electrical insulator organic polymer which is transparent in at least one wavelength range combined with a label emitting at least in the said wavelength range according to the present invention.

Thus, by simple measurement of fluorescence, it is possible to determine whether the object is an authentic object, that is to say comprising the film combined with the label, or whether it is a copy of the said object.

The establishment of an infringement is often difficult, since numerous processes differ both in terms of the treated component and in the nature of the interface which has been constructed between the polymer film and the surface of the untreated component. As this interface is buried beneath the film which has been deposited, it becomes difficult to analyse it through the deposited film, in particular when its thickness is greater than 10 nm. In this case, a proprietor of an intellectual property title relating to a process for the deposition of thin films can advantageously label the films manufactured by him/her with a label, for example a fluorescent, phosphorescent or chemiluminescent label, in accordance with the present invention in a sufficiently low concentration for a measuring device such as that described above to be necessary for its detection.

The abovementioned applications are to be regarded only by way of illustration and neither these applications nor the procedure by which the insulating polymers constituting the film are deposited on the surface of the substrates should constitute a limitation to the application of the present invention.

This is because a person skilled in the art will know how to measure, with regard to other applications, the significance of the present invention by combining one or more organic coating(s) of one or more electrical insulator polymer(s) having low optical extinctions in a wavelength range and a label emitting in the said range.

Other advantages and characteristics of the present invention will become more apparent to a person skilled in the art on reading the examples below, given by way of illustration and without limitation, with reference to the appended figures.

### BRIEF DESCRIPTION OF THE FIGURES

FIG. 1 is a diagram illustrating the principle of the rotating polarizer ellipsometry used to measure the thickness of the films of insulating polymer according to the present invention.

FIGS. 2 and 3 are ellipsometric measurement spectra of gold, as substrate, for different angles of incidence in degrees: tan(Ψ)=f(λ(nm)) (FIG. 2) and cos(Δ)=f(λ(nm)) (FIG. 3) (λ=wavelength in nm).

FIG. 4 is a graph combining measurements of index (I) and (N) and of optical extinction coefficient (E) and (K) carried out on a substrate of gold alone (G) and on a substrate covered with an organic film, measured at an angle of 75° for all the wavelengths between 300 and 800 nm.

FIGS. 5 and 6 are ellipsometric spectra of a platinum substrate without an organic polymer film obtained from measurements carried out over a spectral range extending from 300 to 800 nm with a step of 5 nm: tan(Ψ)=f(λ(nm)) with an incidence of 750 (FIG. 5) and cos(Δ)=f(λ(nm)) with an incidence of 750 (FIG. 6).

FIG. 7 is a graph combining measurements of index (I) and (N) and of optical extinction coefficients (E) and (K) carried out on a platinum substrate without an organic film, measured at an angle of 75° for all the wavelengths between 300 and 800 nm, and values given by the “Handbook of Optical Constants of Solids”, edited by E. D. Palik.

FIGS. 8a), 9a), 10a and 11a) represent ellipsometric spectra of a platinum substrate covered with a PMAN film which are obtained from measurements carried out between 55 and 75° with a step of 5°, the spectral range extending from 300 to 800 nm with a step of 5 nm: tan(Ψ)=f(λ(nm)), for the samples AuMAN7, AuMAN24, Au2401 and Au2301 respectively.

FIGS. 8b), 9b), 10b) and 11b) represent ellipsometric spectra of a platinum substrate covered with a PMAN film obtained from measurements carried out between 50 and 75° with a step of 5°, the spectral range extending from 300 to 800 nm with a step of 5 nm: with an incidence of 75° (FIG. 5) and cos(Δ)=(λ(nm)), for the samples AuMAN7, AuMAN24, Au2401 and Au2301 respectively.

FIG. 12 is a graphical representation of the measurements of the reflection (in %) of the samples with a gold substrate coated with different transparent electrical insulator organic polymer films according to the invention as a function of the wavelength (in nm).

FIGS. 13a) and 13b) are spectra of ellipsometric measurements carried out between 50 and 75° with a step of 5°, the spectral range extending from 300 to 800 nm with a step of 5 nm, on a platinum substrate coated with a film of conducting organic polymer based on diazonium salts (sample 01010Pt6), for different angles of incidence in degrees: tan(Ψ)multiangles=f(λ(nm)) (FIG. 13a)) and cos(Δ)multiangles=f(λ(nm)) (FIG. 13b)).

FIGS. 14a) and b) are spectra of ellipsometric measurements carried out between 50 and 75° with a step of 5°, the spectral range extending from 300 to 800 nm with a step of 5 nm, on a platinum substrate coated with a film of conducting organic polymer based on diazonium salts (sample 01010Pt14), for different angles of incidence in degrees: tan(Ψ)multiangles=f(λ(nm)) (FIG. 14a)) and cos(Δ)multiangles=f(λ(nm)) (FIG. 14b)).

FIG. 15 represents the results of spectrophotometric measurements intended to determine the reflection of a sample of platinum coated with a conducting organic film based on diazonium salts (samples 0101Pt6 and 0101Pt14), over the range 400 to 800 nm with a reference wavelength of 560 nm.

FIG. 16 represents the results of spectrophotometric measurements intended to determine the losses of a sample of platinum coated with a conducting organic film based on diazonium salts (samples 0101Pt6 and 0101Pt14), over the range 400 to 800 nm with a reference wavelength of 560 nm.

FIG. 17 is a diagram for the dipolar modelling of a surface fluorophore: it represents a dipole with a dipole moment m placed above a surface x. Three media are distinguished: the environment of the dipole, the area 1 with an index n₁, in this instance the superstrate; a stack of thin layers, the area 2 with an index n₂; and the substrate, the area 3 with an index n₃.

FIGS. 18a) and 18b) represent modellings (signal (ua)=f(thickness of the polymer) (in nm)) of the fluorescence signal of a fluorophore dipole placed at the surface of an organic film according to two orientations, parallel (//) (FIG. 18a)) and perpendicular (⊥) (FIG. 18b)) to the surface. In these figures, case 1: n=1.5 and k=0.02; case 2: n=1.5 and k=0.003; case 3: n=1.5 and k=0.0 (extreme value); the case 4 models a typical conducting organic film, such as those obtained from diazonium salts: n=1.5 and k=0.4. In these figures, the axis of the abcissae represents “t” the thickness of the polymer film and the axis of the ordinates represents “s” the signal.

FIGS. 19a) and 19b) are diagrammatic representations of gold substrates coated, on the one hand, with a film of polypyrrole (FIG. 19a)) in accordance with the prior art and, on the other hand, with an insulating organic polymer film (ManAu11) (FIG. 19b)) in accordance with the present invention.

FIG. 20a) is a negative (objective ×50; exposure time: 200 ms) of the fluorescence of a drop (0.5 μl of a 1 μM solution) of Cydctp (trade mark) fluorophore from Amersham deposited on a gold substrate, over the gold zone.

FIG. 20b) is a negative (objective ×50; exposure time: 200 ms) of the fluorescence of a drop (0.5 μl of a 1 μM solution) of Cydctp (trade mark) fluorophore from Amersham deposited on a film of polypyrrole deposited on a gold substrate, over the zone corresponding to the polypyrrole.

FIG. 20c) is a negative (objective ×50; exposure time: 200 ms) of the fluorescence of a drop (0.5 μl of a 1 μM solution) of Cydctp (trade mark) fluorophore from Amersham deposited on a film of polymethacrylonitrile (MAN) deposited on a gold substrate (AuMan11 sample), over the zone corresponding to the deposition of MAN.

### EXAMPLES

### Example 1

### Techniques for Measuring and Modelling the Optical Properties of a Sample

The extinction measurements are carried out by spectroscopic ellipsometry. This is a nondestructive optical technique which makes it possible to characterize, by determination of the index and of the thickness, thin deposits of materials by making use of the modifications which the latter produce on the polarization of light.

When a beam is reflected at the surface of a sample, its state of polarization is modified. This is because, at oblique incidence, the electrical field of a light wave is broken down along two specific directions, one of which is perpendicular to the plane of incidence (wave S) and the other parallel to this plane (P). These two waves interact differently with the surface of a sample and reveal amplitude reflection coefficients rₛ and rₚ according to whether they relate to the S or P waves.

The change in state of polarization, which results from the difference in amplitude behaviour and phase behaviour of the S and P waves, can then be characterized by ρ according to the following equation eq1:

$$ρ = \frac{{r}_{P}}{{r}_{S}} = tan ( Ψ ) {ⅇ}^{ⅈ Δ} \qquad eq1$$

tan(Ψ) represents an amplitude ratio and Δ a phase difference between the S and P polarizations.

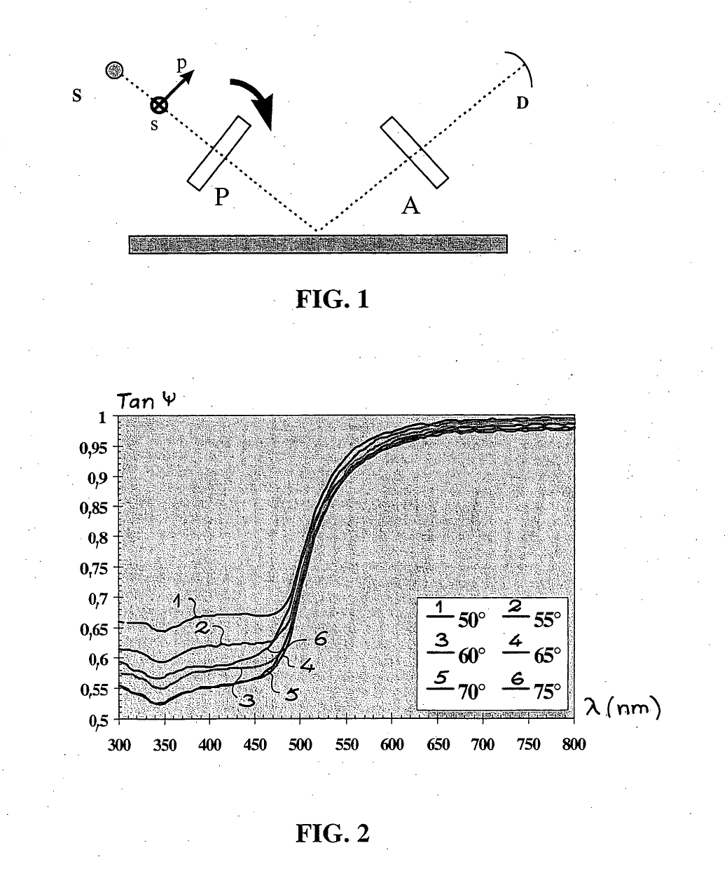

The ellipsometer used is the GESP5 (trade mark) from SOPRA. It makes it possible to measure the parameters tan(Ψ) and cos(Δ) as a function of the wavelength, hence the term “spectroscopic ellipsometry”, and/or of the angle of incidence θ of the analytical light beam. The operating principle of the rotating polarizer ellipsometer used is set out in FIG. 1.

In this figure, S represents a light source, s and p the polarization vectors of the incident light, P and A rotating polarizers and D a detector.

By means of modelling the sample, it is possible to calculate the characteristics of the latter using regression algorithms on the measurements given by the ellipsometer.

The samples, thickness t, index n and extinction k, were characterized by regression of the ellipsometric measurements using smoothing software by nonlinear regression in the complex plane with a nondispersive law and then with a Lorentz oscillators model.

The consistency of the results was confirmed with the spectrophotometric measurements.

### Example 2

### Samples Examined

The measurements were carried out on eight different samples, the characteristics of which are summarized in Table 1.

Three separate series of samples were analysed:
a series of PMAN films electrografted to gold, with a thickness varying between 9 and 150 nm. These films are obtained by electroreduction of a 2.5M solution of methacrylonitrile in anhydrous acetonitrile on a gold electrode in the presence of 0,05 M tetraethylammonium perchlorate (TEAP) as supporting electrolyte. The electroreduction takes place under voltametric conditions between −0.3 and −2.6 V/(Ag⁺/Ag) at 100 mV/s, in nonseparated compartments, with a platinum counterelectrode with a high surface area. The various thicknesses are obtained by varying the number of voltametric sweeps;
two PMMA films electrografted to gold, one with a thickness of 100 nm, the other thick (thickness >0.5 μm). These films are obtained by electroreduction of a methyl methacrylate solution under the same conditions as for the PMAN films;
two films obtained by electroreduction of a 10⁻³M solution of para-nitrophenyldiazonium (PNPD) tetrafluoroborate in anhydrous acetonitrile on a platinum electrode, in the presence of 5×10−²M TEAP as supporting electrolyte. The potential sweeps are applied from +0.3 V/(Ag⁺/Ag) to −2.9 V/(Ag⁺/Ag), at −200 mV/s. Two films with respective thicknesses of 3 and 30 nm are obtained by this protocol.

This final series of samples was chosen as the organic films obtained by electrografting PNPD are electrically conducting, in contrast to the films of the first two series. In addition, the ellipsometric measurements will also be compared to those obtained on a polypyrrole film as conducting polymer.

The thicknesses of the various films listed in Table I below are measured by profilometry. The arithmetic roughnesses, measured by profilometry, according to two tip distances: 500 μm and 2 mm, are also listed in this table.

TABLE I
References and characteristics of the samples used in the
implementational examples

| | | | Thick- | Ra at | Ra at |
|---|---|---|---|---|---|
| | Sub- | | ness | 500 μm | 2 mm |
| Name | strate | Coating | (nm) | (nm) | (nm) |
| MANAu 15 | Gold | Polymethacrylonitrile | 9 | 4 | 0.7 |
| MANAu 24 | Gold | Polymethacrylonitrile | 28 | 3.9 | 6.5 |
| Au MAN 11 | Gold | Polymethacrylonitrile | 50 | 2.9 | 2.9 |
| Au MAN 7 | Gold | Polymethacrylonitrile | 150 | 3.4 | 5.3 |
| Au 2301 | Gold | Polymethacrylonitrile | 100 | — | — |
| Au 2401 | Gold | Polymethylmethacrylate | Thick | — | — |
| 0101 Pt 14 | Pt | Nitrobenzoic | 3 | 6.5 | 8.3 |
| 0101 Pt 6 | Pt | Nitrobenzoic | 30 | 2.4 | 2.4 |

In this table, the polymethacrylonitrile or poly(methyl methacrylate) films are insulating and the nitrobenzoic films are conducting.

ELLIPSOMETRIC MEASUREMENTS ON THE SUBSTRATES

Characteristics of the Gold Layer (Substrate)

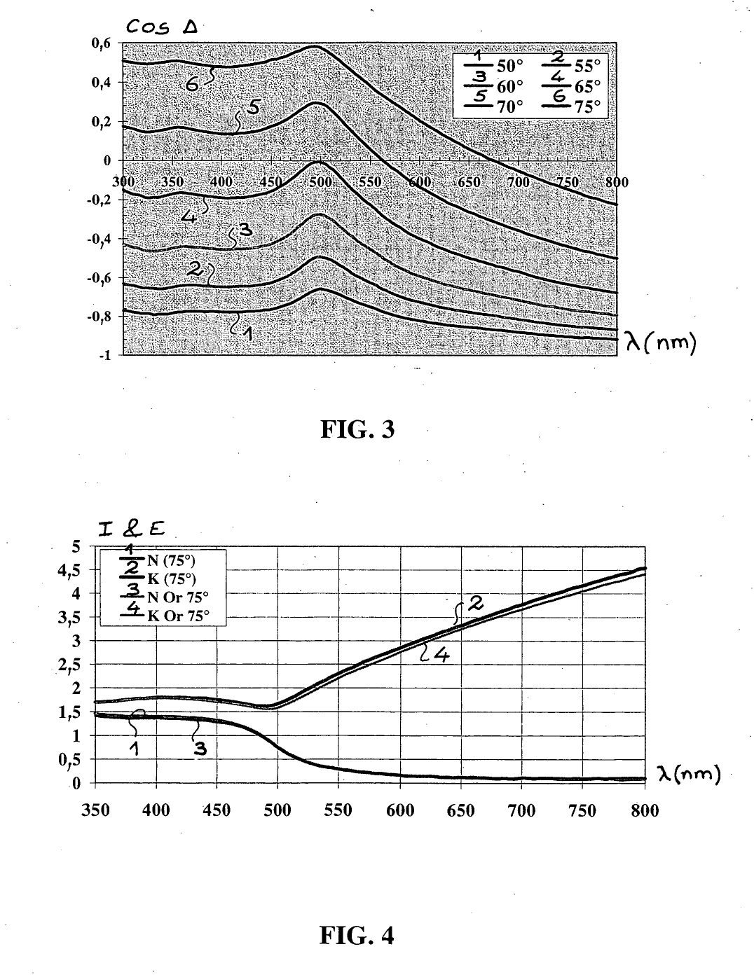

The ellipsometric spectra of the gold are measured for different angles of incidence. In this way, it is possible to assess the quality of the final result as a function of the results obtained for these various values, between 50° and 75° with a step of 5°. The measurement spectra extend between 300 and 800 nm with a measurement step of 5 nm. They are presented in the appended FIGS. 2 and 3.

The measurement of the gold was carried out on the MANAu11 sample, on the part not dipped in the reaction mixture and on which no organic film has been deposited.

The aim is to find the values of index and of extinction of this gold layer; its thickness of greater than a micron makes it possible to put it in the same category (ellipsometric) as a substrate. An inversion, equation resolution, of the data is then carried out in order to extract the n and k coefficients of the gold. This operation is carried out for each measurement angle. The indices measured at an angle of incidence of 75° have been listed in the appended FIG. 4 for all the wavelengths between 300 and 800 nm.

For comparison, the measurements carried out on a substrate of gold alone have also been listed, independently of the samples carrying a grafted organic film, which makes it possible to monitor the stability of the measurements.

Characteristics of the Platinum Layer (Substrate)

The ellipsometric spectra of platinum are measured under the same conditions as for gold: measurement angle between 50° and 75° with a step of 5° and the spectral range extends from 300 to 800 nm with a step of 5 nm.

The measurement of the platinum was carried out on the 0101Pt6 sample, on the part not dipped in the reaction mixture and on which no organic film has been deposited.

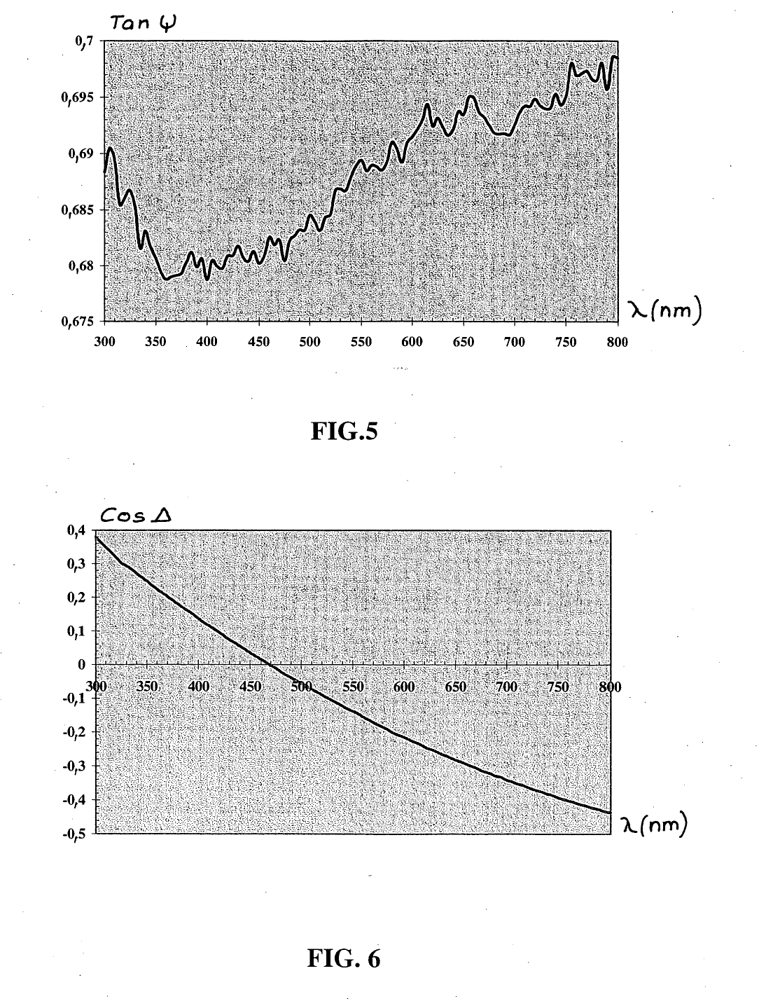

FIGS. 5 ((tanΨ)=f(λ(nm)), incidence 75°) and 6 ((CosΔ)=f(λ(nm)), incidence 75°) are graphical representations of the results obtained for these measurements.

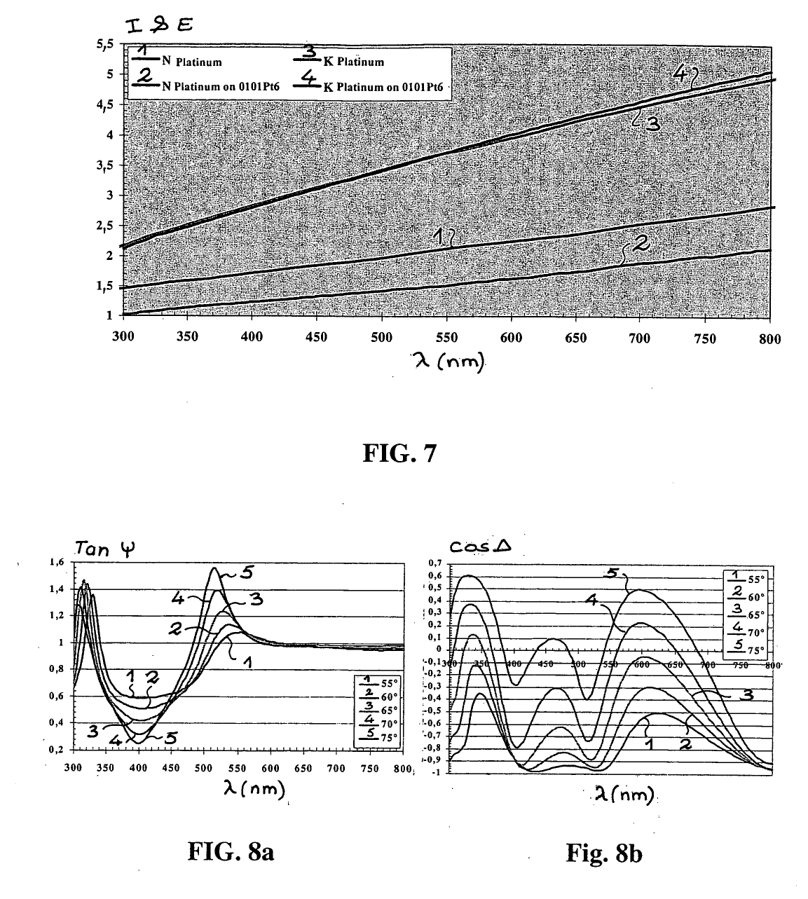

The aim is to find the values of index and extinction of this platinum layer: its thickness of greater than one micron allows it to be put into the same category, from an ellipsometric viewpoint, as a substrate. An inversion of the data of the measurement at 75° is then carried out in order to extract therefrom the n (index) and the k (extinction coefficient). The results of index (I) and extinction (E) thus obtained are combined in FIG. 7. Values given by the “Handbook of Optical Constants of Solids”, edited by E. D. Palik, have also been represented in this graph.

For the characterizations of the samples, the results given by the ellipsometric measurement will be taken as reference values for platinum.

ELLIPSOMETRIC MEASUREMENTS OF THE SAMPLES

The measurements are carried out over the spectral range 300-800 nm and at θ=60°.

Ellipsometric Results of the Samples on a Gold Substrate

In order to be more exact with regard to the results, the AuMAN7, MANAu24, Au2301 and Au2401 samples were measured for variable angles.

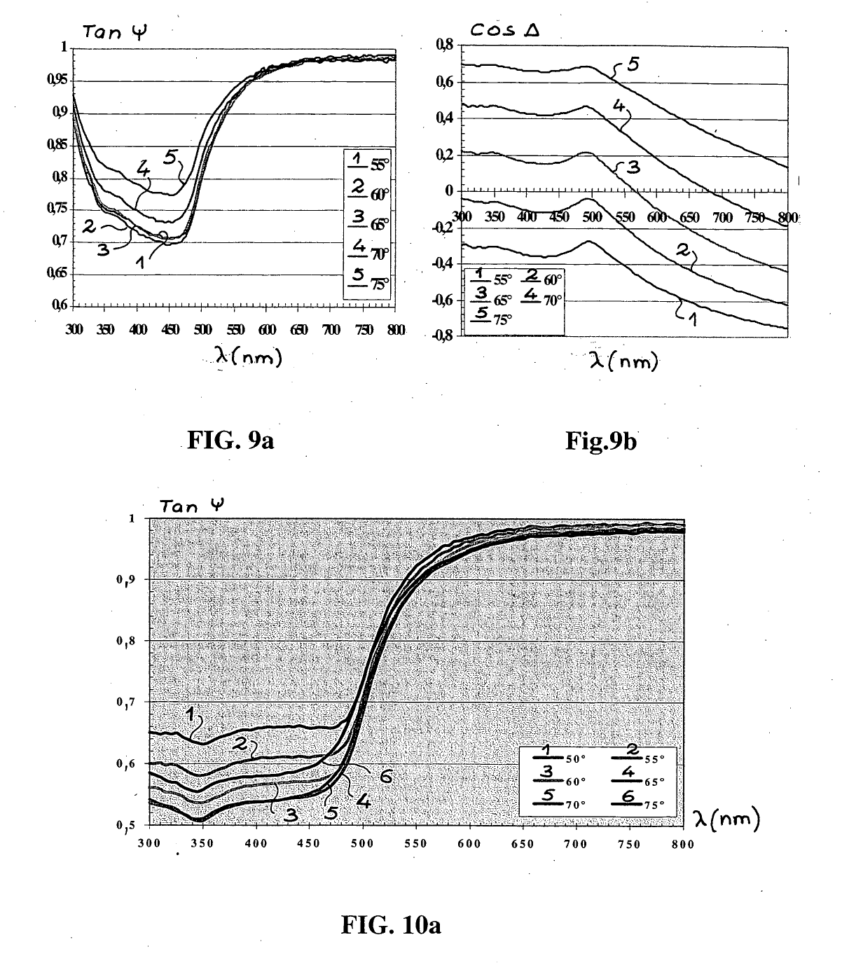

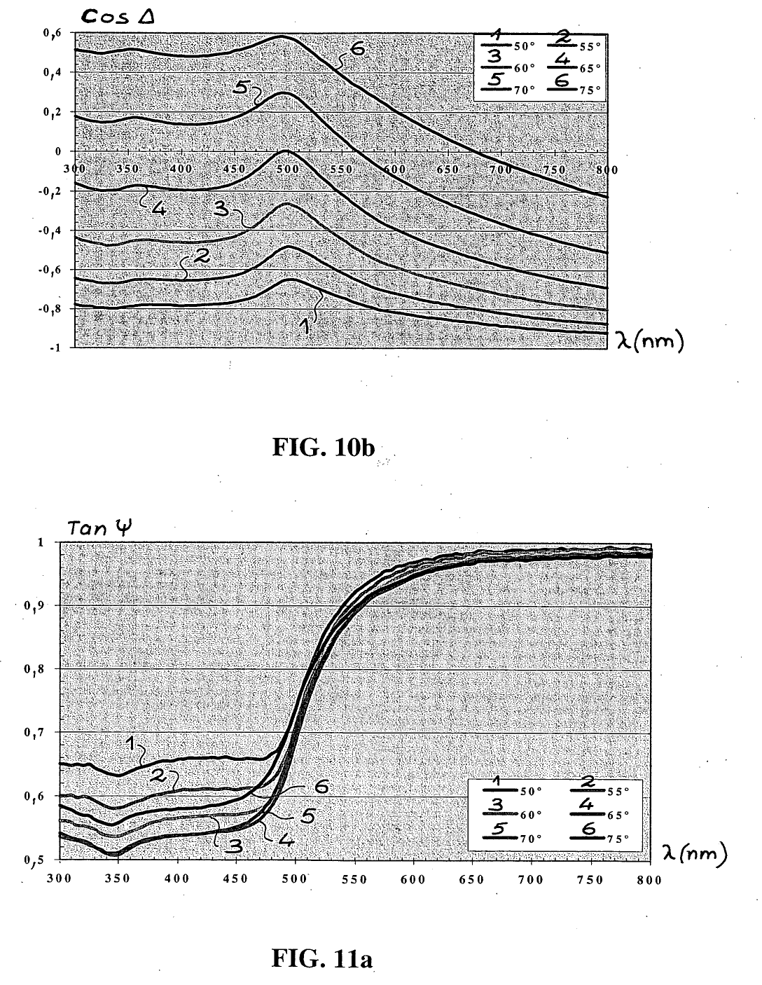

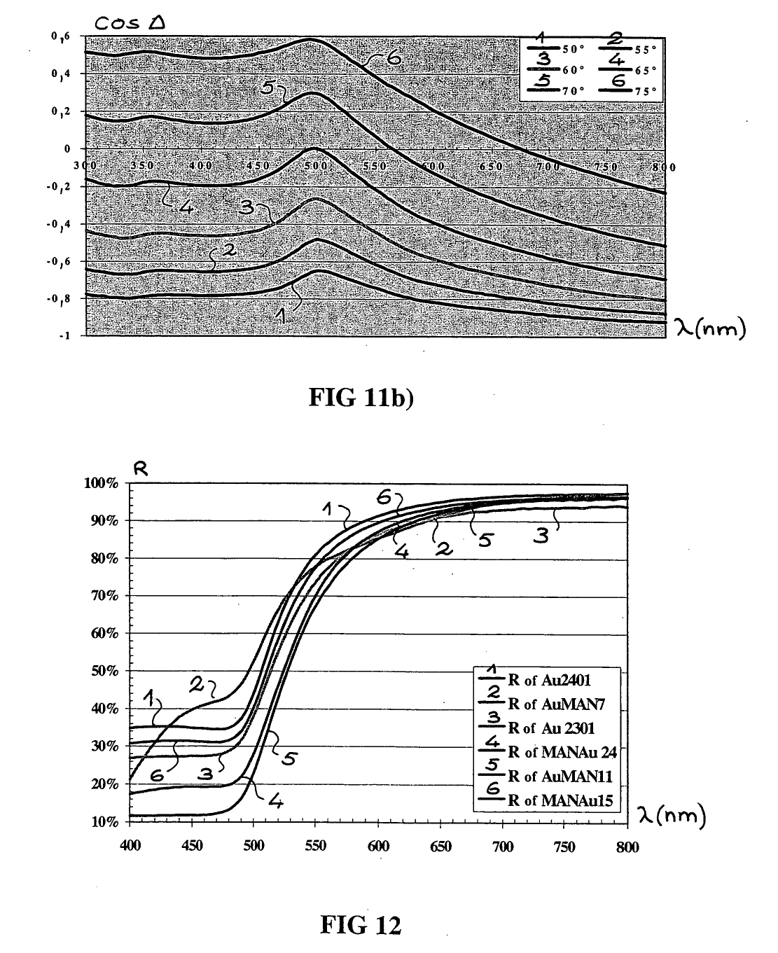

The measurements carried out have made it possible to put together the spectra represented in FIGS. 8a) and b) for AuMAN7; in FIGS. 9a) and b) for AuMAN24; in FIGS. 10a) and 10b) for Au2401; and in FIGS. 11a) and b) for Au2301. The a) figures correspond to the tan(Ψ)multiangle measurements and the b) figures correspond to the cos(Δ)multiangle measurements.

Spectrophotometric Results of the Samples on the Gold Substrate

The samples were measured with a manual “Lambda9m” (trade mark) spectrophotometer in order to determine the Reflection, the Transmission and the Losses of each of them over the wavelength range 400-800 nm, with 560 nm as reference wavelength.

It is found that all the samples on gold have the same spectra and that, at 560 nm, 72.5%<R<84.2%, T=0 and 157%<L<27.5%. These results are combined in Table 2 below:

TABLE 2
Reflection (R), Transmission (T) and Losses (L) at 560 nm for the
samples on gold

| | at 560 nm | | |
|---|---|---|---|
| Sample | R(%) | T(%) | L(%) |
| MANAu 11 | 72.55 | 0 | 27.46 |
| MANAu 15 | 81.32 | 0.02 | 18.66 |
| MANAu 24 | 75.45 | 0.03 | 24.51 |
| AuMAN 7 | 79.96 | 0 | 20.05 |
| Au 2301 | 77.54 | 0.0207 | 22.44 |
| Au 2401 | 84.19 | 0.04 | 15.77 |

The appended FIG. 12 is a graphical representation of the measurements of the reflection (in %) carried out on these samples comprising a gold substrate coated with different transparent and electrical insulator organic polymer films according to the invention as a function of the wavelength (in nm).

It is found that all the samples on gold have the same spectra and that, at 560 nm, 72.5%<R<84.2%, T=0 and 15.7%<L<27.5%. It is found in particular that these insulating films have few losses over a wide wavelength range. This observation will become even clearer on comparing the results obtained with the conducting PNPD films.

Ellipsometric Results of the Samples on a Platinum Substrate

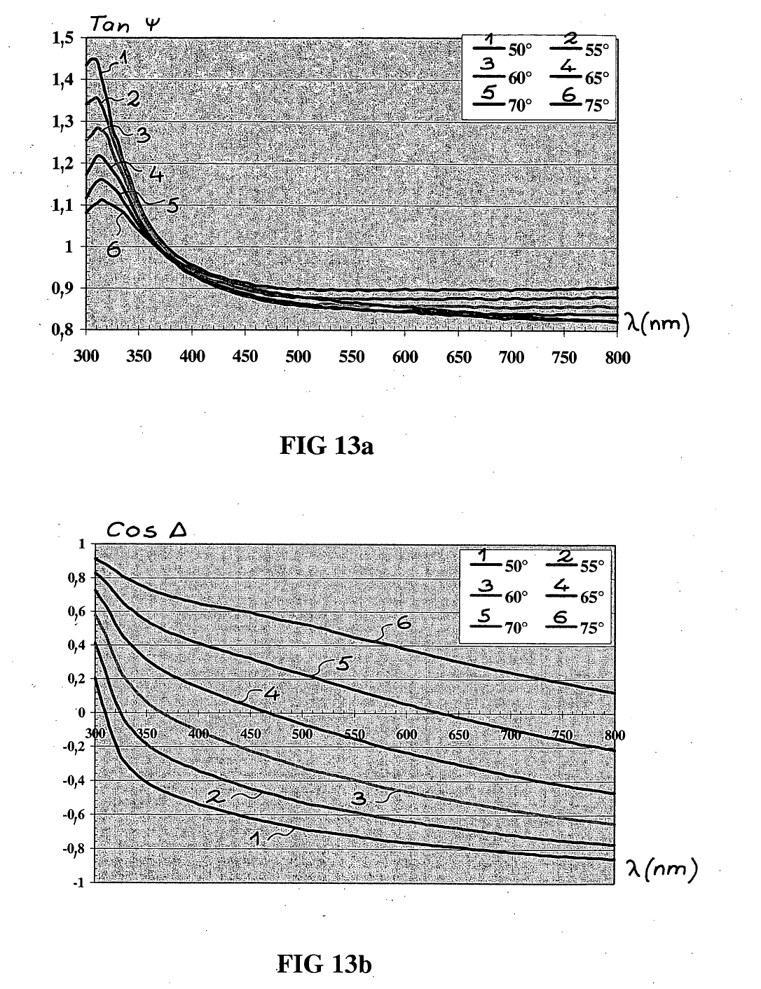

FIGS. 13a) and 13b) represent the ellipsometric measurement spectra recorded between 50 and 75° with a step of 5°, the spectral range extending from 300 to 800 nm with a step of 5 nm, on the 0101Pt6 samples for various angles of incidence in degrees: tan(Ψ)multiangles=f(λ(nm)) (FIG. 13a)) and Cos(Δ)multiangles=f(λ(nm)) (FIG. 13b)).

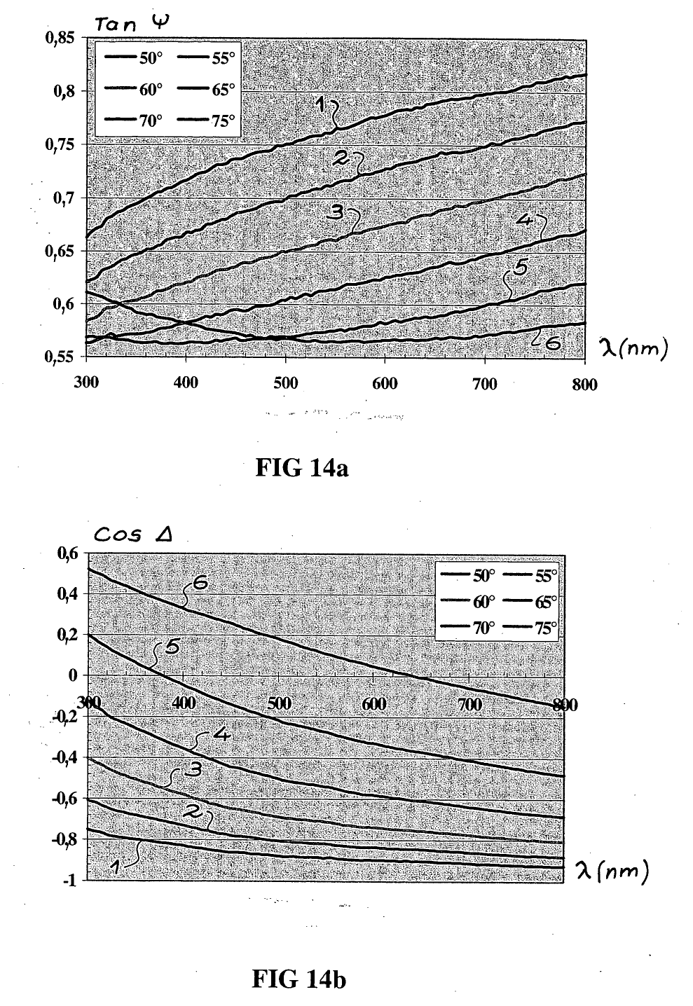

FIGS. 14a) and 14b) represent the ellipsometric measurement spectra recorded between 50 and 75° with a step of 5°, the spectral range extending from 300 to 800 nm with a step of 5 nm, on the 0101Pt14 samples for various angles of incidence in degrees: tan(Ψ)multiangles=f(λ(nm)) (FIG. 14a)) and Cos(Δ)multiangles=f(λ(nm)) (FIG. 14b)).

Spectrophotometric Results of the Samples on a Platinum Substrate

The samples were measured with a manual “Lambda9m” (trade mark) spectrophotometer in order to determine the Reflection, the Transmission and the Losses of each of them over the wavelength range 400-800 nm, with 560 nm as reference wavelength.

It is found that the samples on platinum have very different spectra. The reflection, transmission and losses are summarized in Table 3 below. It is observed that the losses in the PNPD films, PNPD being conducting polymers, are much more significant than in the case of insulating films produced on gold substrates.

TABLE 3
Reflection (R), Transmission (T) and Losses (L) at 560 nm for the
samples on platinum

| | at 560 nm | | |
|---|---|---|---|
| Sample | R(%) | T(%) | L(%) |
| 0101Pt14 | 45.72 | 0.02 | 54.26 |
| 0101Pt6 | 57.32 | 0.02 | 42.66 |

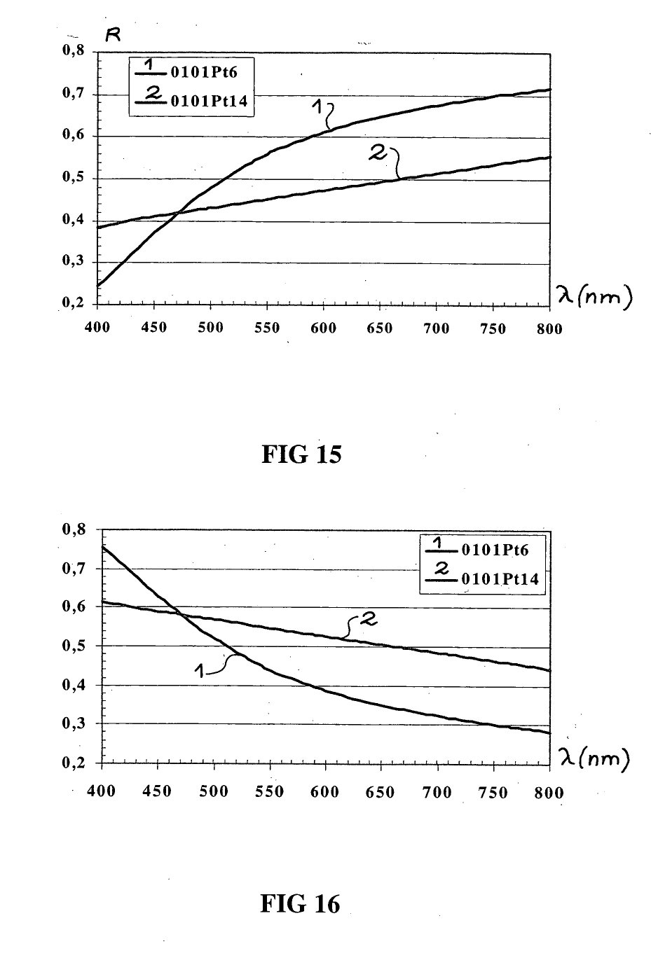

FIG. 15 represents the reflection and FIG. 16 represents the losses of the platinum samples coated with a conducting polymer 0101Pt6 and 0101Pt14 over the range 400 to 800 nm with a reference wavelength of 560 nm.

CHARACTERIZATION OF THE SAMPLES

To characterize the samples (t, n and k), regression was carried out on the ellipsometric measurements using SporX (trade mark) software with a nondispersive law and then with Lorentz oscillators. Consistent results were obtained, on the one hand between the two models and, on the other hand, with the spectrophotometric measurements.

The assessment of these characterizations is summarized in Table 4.

Very low extinction coefficients are measured for the insulating vinyl films (k<0.02), whereas the conducting PNPD films give extinctions which are at least 10 times higher.

By way of comparison, the polypyrrole films with a thickness similar to those of the samples measured (20 nm) have an extinction coefficient of 0.3 to 0.5, measured under the same conditions.

TABLE 4
Optical characteristics of the samples

| | | t | t | | |
|---|---|---|---|---|---|
| | | (profilo) | (ellipso) | | |
| Sample | Nature | nm | nm | n | k |
| MANAu15 | insulating | 9 | 5-6 | 1.5 | 0.02 |
| MANAu24 | insulating | 28 | 24 | 1.5 | 0 |
| AuMAN11 | insulating | 50 | 40-46 | 1.5-1.6 | 0.003 |
| AuMAN7 | insulating | 150 | 149 | — | — |
| Au2301 | insulating | 100 | — | 1.1 | 0.02 |
| Au2401 | insulating | (thick) | — | 1 | 0 |
| 0101Pt6 | conducting | 3 | — | 1.9 | 0.1 |
| 0101Pt14 | conducting | 30 | 25-30 | 0.9 | 0.5 |

The aim now is to characterize the signal which would be emitted by a fluorophore adsorbed on the surface of the film.

First of all, the expected advantage of the fact of the low extinctions is presented and then it is shown, on a simple test, that the fluorescence yield is effectively better on the PMAN films on gold than on a polypyrrole film of the same thickness.

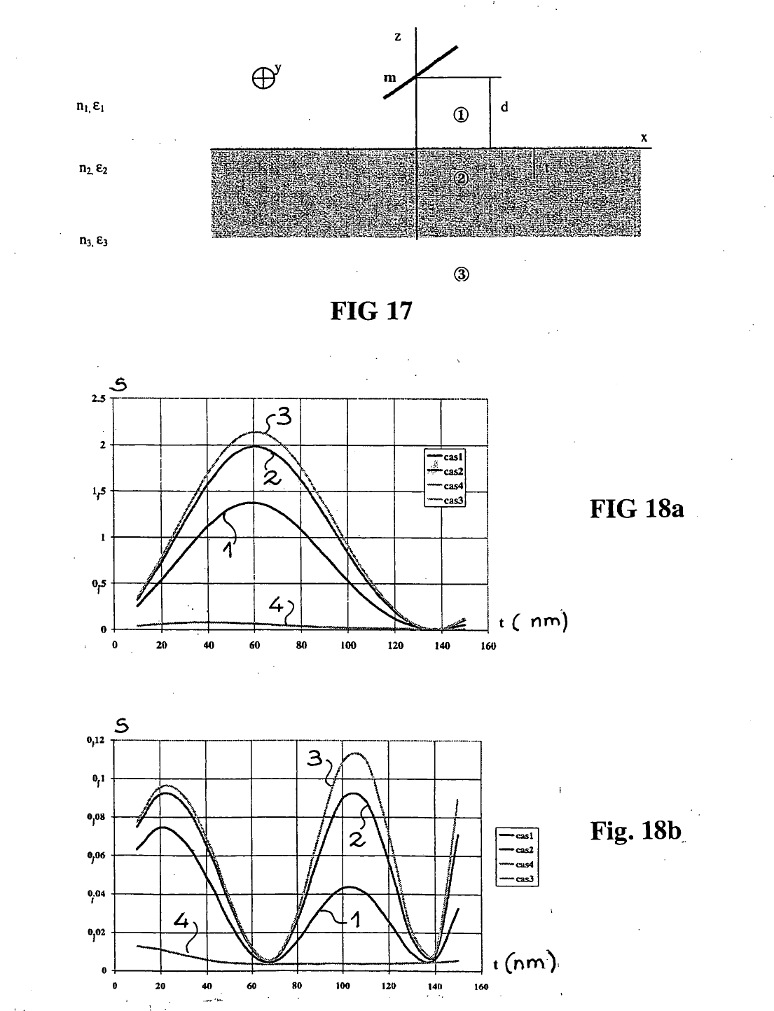

The fluorophore is modelled as being an electric dipole, as represented in FIG. 17, that is to say the small source which can be envisaged in the context of the electromagnetic theory. This step is not in any way harmful to the general application of the question treated, since the Green's function of the problem can be calculated and any more complex electromagnetic source, such as superposition of base dipoles, can be reconstructed. Furthermore, it is assumed that the electromagnetic fields will not be quantized. The comparison of theoretical models for modelling the fluorescence and the luminescence in laser cavities has shown near-similar results, whether the viewpoint is classical or quantum.

On the basis of the results of Table 4, the following cases are simulated: case 1: n=1.5/k=0.02; case 2: n=1.5/k=0.003; case 3: n=1.5/k=0 (extreme value); the final case models a typical conducting organic film, such as, for example, the films obtained from diazonium salts (samples 0101Pt6 and 0101Pt14) case 4: n=1.5/k=0.4.

The fluorophore used is phycoerythrin, absorption=543 nm and emission=580 nm; its altitude with respect to the surface is 10 nm. The fluorophore is immersed in a liquid environment.

Two different orientations are taken into account for the dipoles: one parallel and the other perpendicular to the surface. The thickness of the polymer is varied and the signal emitted into the medium surrounding the fluorophore is calculated (case of a very open microscope for the analysis of fluorescence).

FIG. 17 is a diagram of the dipolar modelling of a fluorophore at the surface: it represents a dipole with a dipole moment m placed above a surface x. Three media are distinguished: the environment of the dipole, the area 1, in this instance the superstrate; a stack of thin layer(s), the area 2; and the substrate, the area 3. n₁, ε₁; n₂, ε₂ and n₃, ε₃ are respectively the index and the dielectric permittivity of the areas 1, 2 and 3; d represents the distance from the dipole to the surface of the polymer and t represents the thickness of the polymer.

FIGS. 18a) and 18b) represent modellings (signal (ua)=f(thickness of the polymer) (in nm)) of the fluorescence signal of a fluorophore dipole placed at the surface of an organic film according to two orientations, parallel (FIG. 18a)) and perpendicular (FIG. 18b)) to the surface. In these figures, case 1: n=1.5 and k=0.02; case 2: n=1.5 and k=0.003; case 3: n=1.5 and k=0.0 (extreme value); the case 4 models a typical conducting organic film, such as, for example, the films obtained from diazonium salts (samples 0101Pt6 and 0101Pt14): n=1.5 and k=0.4.

In the light of the results represented in the appended FIGS. 18a) and 18b), an increase of approximately 25 with regard to the signal is observed with the present invention using a “MAN” film with a parallel orientation of fluorophores, the most probable case because of the phenomenon of photoselection, and of approximately 100 for a perpendicular orientation.

The predictions of this model are now tested in a simple way by examining the fluorescence signal of a drop of fluorescent label on a film of PMAN (AuMAN11 sample) deposited on a gold substrate and by comparing it with the same signal with regard to a polypyrrole film with the same thickness deposited on a gold substrate. The fluorophore employed is 1 μM Cy₃dctp (trade mark) (Amersham). The volume of the drops is 0.5 μl.

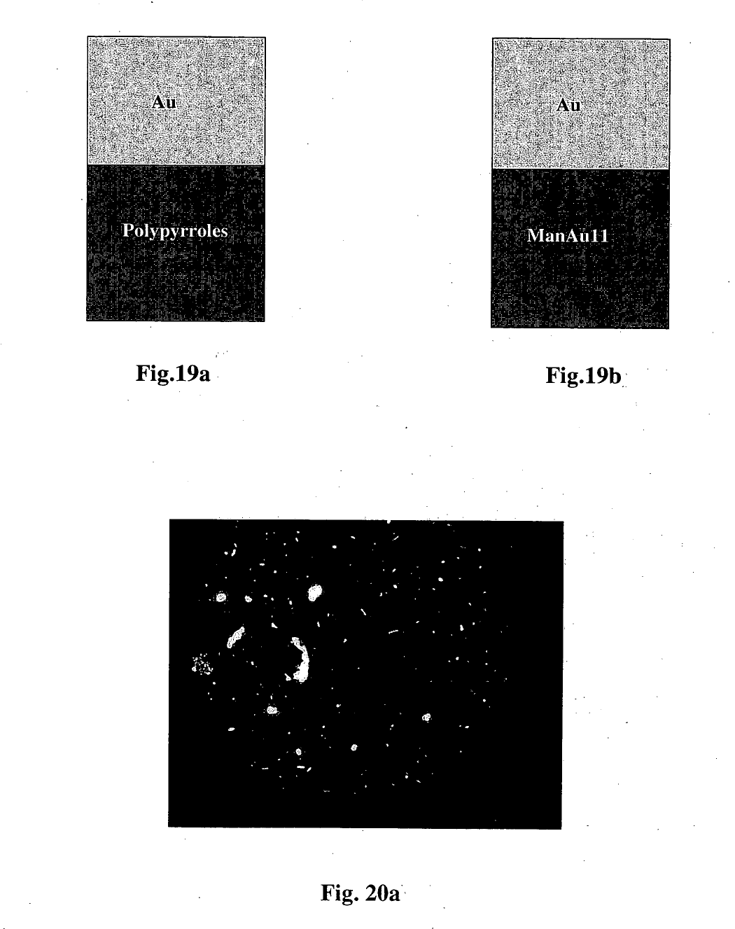

FIGS. 19a) and 19b) are diagrammatic representations of the gold substrates coated, on the one hand, with a film of polypyrrole (FIG. 19a)) and, on the other hand, with an insulating organic polymer film (ManAu11) (FIG. 19b)) in accordance with the present invention.

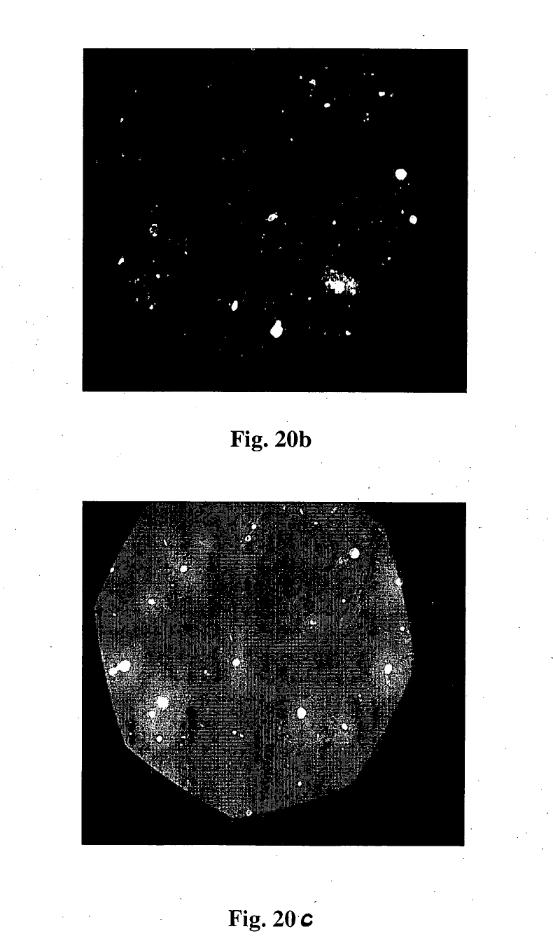

FIGS. 20a), b) and c) are negatives (objective ×50; exposure time: 200 ms) of the fluorescence on the area of gold (FIG. 20a)), on the film of polypyrrole (FIG. 20b)) and on the film of polymethacrylonitrile (MAN) which are obtained in this example.

The highly luminous points on the three photographs are either agglomerates of fluorophores, or fluorescent dust. These points do not correspond to fluorophores close to the surface, that is to say a few nm, which is the case with biochips, for example, but to balls from a few tens to a few hundreds of nm. It is therefore not with regard to these light points that the comparison has to be carried out but with regard to the “continuous background” which surrounds these points.

It is observed that, with identical exposure times, photographic receptors of the same sensitivity, the same magnifications achieved with the same optics, and even though the concentration of label is very low (1 μm), the fluorophores which are in “direct” contact with the surface are not “extinguished” in the case of the AuMAN11 samples (negative c)), whereas they are extinguished over the areas of gold (negative a)) and over the area of the conducting organic coating (negative b)). Thus, in this instance, the superior properties of transparency of the insulating films of PMAN with regard to the conducting films are directly observed, as illustrated in these FIG. 20.

### Example 3

### Production of a Deposit of Polymethacrylonitrile Film by Electroinitiation in the Presence of Diazonium Salts

A deposit of polymethacrylonitrile films is produced, on the same gold surfaces as those of Example No. 1, by electroinitiation starting from diazonium salts. These films are obtained by electroreduction of a 2.5M solution of methacrylonitrile in anhydrous acetonitrile in the presence of 0,05 M tetraethylammonium perchlorate (TEAP) as supporting electrolyte and of 10⁻³M 4-nitrophenyldiazonium tetrafluoroborate. The electroreduction takes place under voltametric conditions between +0.3 and −1.5 V/(Ag⁺/Ag) at 100 mV/s, in nonseparated compartments, with a platinum counterelectrode with a high surface area. The number of sweeps is adjusted so as to obtain films with a thickness of the order of 100 nm.

The optical measurements reveal a coefficient k<0.02, as observed with the polymethacrylonitrile films obtained by direct electrografting (cf. Table 4).

### Example 4

### Procedure for Monitoring Reproducibility

Polymethacrylonitrile films of variable thickness peppered with phycoerythrin are manufactured on entirely identical gold strips. For this, the gold strips are polarized under voltametric conditions between +0.3 and −1.0 V/(Ag⁺/Ag) at a 100 mV/s, in nonseparated compartments, with a platinum counterelectrode with a high surface area, in a solution containing 2.5M methacrylonitrile in anhydrous acetonitrile, 0,05 M tetraethylammonium perchlorate (TEAP) as supporting electrolyte, 10⁻³M 4-nitrophenyldiazonium tetrafluoroborate and 1 μM phycoerythrin. The thickness of the films obtained, measured by ellipsometry, is 50±5 nm, i.e. an accuracy of 10%.

Each strip is subsequently irradiated at 543 nm and the resulting intensity of fluorescence is measured at 580 nm. Identical intensities of fluorescence are measured on all the strips, with a dispersion of the measurements of less than 15%.

### Example 5

### Quality Control Procedure

PMAN (polymethacrylonitrile) films of increasing thickness are deposited according to the procedure of Example 4 on 1 cm² gold strips. The various thicknesses, between 5 and 150 nm, are obtained by varying the number of voltametric sweeps.

The fluorescence measurement on the coatings obtained makes it possible to plot a calibration curve giving the thickness as a function of the intensity/cm².

A PMAN deposit with a thickness of 84 nm (measurement by ellipsometry) is subsequently produced on a 5 cm² gold surface. The fluorescence measurement indicates, from the calibration curve, a thickness of 75 nm, which makes it possible to validate proper control without directly measuring the thickness.

### LIST OF REFERENCES

[1] Arwin et al., Synthetic Metals, 6, 1983: “Dielectric Function of Thin Polypyrrole and Prussian Blue Films by Spectroscopic Ellipsometry”.
[2] Kim et al., Journal of the Electrochemical Society, 138(11), 1991: “Real Time Spectroscopic Ellipsometry: In Situ Characterization of Pyrrole Electropolymerization”.
[3] Kim et al., Bulletin of the Korean Chemical Society, 17(8), 1996: “Polypyrrole Film Studied by Three-Parameter Ellipsometry”.
[4] Guedon et al., Analytical Chemistry, 22, 6003-6009, 2000: “Characterization and Optimization of a Real-Time, Parallel, Label-Free, Polypyrrole-Based DNA Sensor by Surface Plasmon Resonance Imaging”.

1-16. (canceled)

17. A substrate coated with a film, wherein the film is an electric insulator organic polymer which is transparent in at least one wavelength range and wherein the film is combined with a label which emits at least in the wavelength range.

18. The substrate of claim 17, wherein the film comprises an insulating polymer selected from vinyl polymers.

19. The substrate of claim 17, wherein the film comprises an insulating polymer selected from the group consisting of crosslinked or noncrosslinked polymers of acrylonitrile, methacrylonitrile, methyl methacrylate, ethyl methacrylate, propyl methacrylate, butyl methacrylate, hydroxyethyl methacrylate, hydroxypropyl methacrylate, cyano acrylate, acrylic acid, methacrylic acid, styrene, para-chlorostyrene, N-vinylpyrrolidone, and vinyl halides.

20. The substrate of claim 17, wherein the film comprises a crosslinked or noncrosslinked insulating polymer selected from the group consisting of polyacrylamides, polymers of isoprene, of ethylene, of propylene, of ethylene oxide and molecules comprising strained rings, of lactic acid and of its oligomers, of lactones, of ε-caprolactone, of glycolic acid and of aspartic acid, polyamides, polyurethanes, parylene and polymers based on substituted parylene, oligopeptides and proteins, and the prepolymers, macromers and telechelics based on these polymers, and the copolymers and/or blends which can be formed from the monomers of these polymers and from these polymers themselves.

21. The substrate of claim 17, wherein the film is a vinyl polymer obtained by polymerization of a monomer of formula (I): 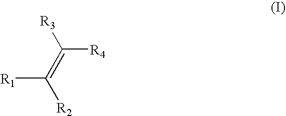
in which R₁, R₂, R₃ and R₄ are independently selected from the group consisting of hydrogen atoms, alkanes, alkenes, alkynes, amides, aldehydes, ketones, carboxylic acids, esters, acid halides, anhydrides, nitriles, amines, thiols and phosphates, ethers, homo- and heterocyclic aromatics and any cyclic group comprising these functional groups, and any group carrying several of these functional groups;
or of a mixture of different monomers of formula (I).

22. The substrate of claim 17, wherein the film is polymethacrylonitrile or poly(methyl methacrylate).

23. The substrate of claim 17, wherein the label is selected from the group consisting of a fluorescent label, a phosphorescent label, and a chemiluminescent label.

24-33. (canceled)

34. A process for the detection of a chemical entity comprising:
providing a substrate coated with a film, wherein the film is an electric insulator organic polymer which is transparent in at least one wavelength range, wherein the film is functionalized with recognition molecules for the chemical entity to be detected;
providing a sample to be analyzed, the sample being labelled with a label which emits at least in the wavelength range;
bringing the sample into contact with the substrate such that the chemical entity to be detected will pair with the recognition molecules of the film; and
detecting for the label.

35. The process of claim 34, wherein the chemical entity to be detected is DNA.

36. The process of claim 34, wherein the label is selected from the group consisting of a fluorescent label, a phosphorescent label, and a chemiluminescent label.

37. A biochip comprising:
a substrate having deposited thereon a film comprising an electrical insulator organic polymer functionalized by a biological probe capable of bringing about pairing with an entity functionalized with a fluorescent, phosphorescent or chemiluminescent label,
wherein the film is transparent in at least one wavelength range suitable for detection of the fluorescent, phosphorescent or chemiluminescent label.

38. The biochip of claim 37, wherein the film is a film of a vinyl polymer.

39. The biochip of claim 37, wherein the film is a vinyl polymer obtained by polymerization of a monomer of the following formula (I): 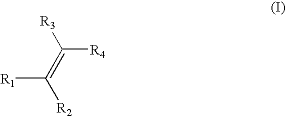
in which R₁, R₂, R₃ and R₄ are independently selected from the group consisting of hydrogen atoms, alkanes, alkenes, alkynes, amides, aldehydes, ketones, carboxylic acids, esters, acid halides, anhydrides, nitrites, amines, thiols and phosphates, ethers, homo-and heterocyclic aromatics and any cyclic group comprising these functional groups, and any group carrying several of these functional groups;
or of a mixture of different monomers of formula (I).

40. The biochip of claim 37, wherein the biological probe is DNA.

41. The biochip of claim 37, wherein the entity functionalized with a fluorescent, phosphorescent or chemiluninescent label is a chemical, biochemical or biological entity.

42. The biochip of claim 41, wherein the entity functionalized with a fluorescent, phosphorescent or chemiluminescent label is a chemical entity.

43. The biochip of claim 42, wherein the chemical entity is DNA.

44. The biochip of claim 41, wherein the entity functionalized with a fluorescent, phosphorescent or chemiluminescent label is a biological entity.

45. The biochip of claim 41, wherein the entity functionalized with a fluorescent, phosphorescent or chemiluminescent label is a nucleic acid, a protein, an antigen, an antibody or a synthetic organic molecule.

46. A process for the detection of a sample, comprising
providing a biochip comprising a substrate having deposited thereon a film having a thickness of less than one micrometer which is an electrical insulator organic polymer which is transparent in at least one wavelength range;
providing a sample;
labeling the sample with a label which emits at least in the one said wavelength range; and
detecting the sample with the biochip.

47. The process of claim 46 wherein the film is a film of a vinyl polymer.

48. The process of claim 46 wherein the film is a vinyl polymer obtained by polymerization of a monomer of the following formula (I): 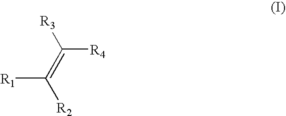
in which R₁, R₂, R₃ and R₄ are independently selected from the group consisting of hydrogen atoms, alkanes, alkenes, alkynes, amides, aldehydes, ketones, carboxylic acids, esters, acid halides, anhydrides, nitriles, amines, thiols and phosphates, ethers, homo-and heterocyclic aromatics and any cyclic group comprising these functional groups, and any group carrying several of these functional groups;
or of a mixture of different monomers of formula (I).

49. The process of claim 46 wherein the electrical insulator organic polymer is functionalized by a biological probe capable of bringing about pairing with a chemical, biochemical or biological entity functionalized with a fluorescent, phosphorescent or chemiluminescent label.

50. The process of claim 49 wherein the sample is labeled with a label which is selected from the group consisting of a fluorescent label, a phosphorescent label, and a chemiluminescent label.

51. The process of claim 46 wherein the biological probe is DNA.

52. A process for the detection of a chemical entity comprising:
providing a film comprising an electrical insulator organic polymer which is transparent in at least one emission wavelength range;
providing a sample of a chemical entity to be detected, the sample being labeled with a label which emits in at least the one emission wavelength range; and
detecting for the label of the sample of the chemical entity.

53. The process of claim 52, wherein the film comprising an electrical insulator organic polymer is functionalized with recognition molecules for the chemical entity.

54. The process of claim 52, wherein the chemical entity is DNA.

55. The process of claim 52, wherein the film is a vinyl polymer obtained by polymerization of a monomer of the following formula (I): 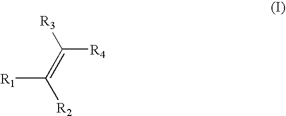
in which R₁, R₂, R₃ and R₄ are independently selected from the group consisting of hydrogen atoms, alkanes, alkenes, alkynes, amides, aldehydes, ketones, carboxylic acids, esters, acid halides, anhydrides, nitrites, amines, thiols and phosphates, ethers, homo-and heterocyclic aromatics and any cyclic group comprising these functional groups, and any group carrying several of these functional groups;
or of a mixture of different monomers of formula (I).

56. The process of claim 52, wherein the film is of polymethacrylonitrile or of poly(methylmethacrylate).

57. The process of claim 52, wherein the label is selected from the group consisting of a fluorescent label, a phosphorescent label and a chemiluminescent label.

58. A process for the detection of a chemical entity comprising:
providing a means for the detection of the chemical entity comprising a film comprising an electrical insulator organic polymer which is transparent in at least one emission wavelength range;
providing a sample of a chemical entity to be detected, the sample being functionalized with a label which emits in at least the one emission wavelength range; and
detecting for the label of the sample of the chemical entity.

59. The process of claim 58, wherein the detection means is a biochip.

60. The process of claim 58, wherein the film is a vinyl polymer obtained by polymerization of a monomer of the following formula (I): 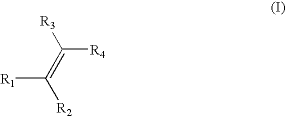
in which R₁, R₂, R₃ and R₄ are independently selected from the group consisting of hydrogen atoms, alkanes, alkenes, alkynes, amides, aldehydes, ketones, carboxylic acids, esters, acid halides, anhydrides, nitrites, amines, thiols and phosphates, ethers, homo-and heterocyclic aromatics and any cyclic group comprising these functional groups, and any group carrying several of these functional groups;
or of a mixture of different monomers of formula (I).

61. The process of claim 58, wherein the film is of polymethacrylonitrile or of poly(methylmethacrylate).

62. The process of claim 58, wherein the label is selected from the group consisting of a fluorescent label, a phosphorescent label and a chemiluminescent label.

63. A process for quality control, certification or authentication of an object comprising:
obtaining the object by combining a film comprising an electrical insulator organic polymer which is transparent in at least one emission wavelength range with a label which emits in at least the one emission wavelength range; and
measuring the intensity of the emission of the at least one wavelength range of the object.

64. The process of claim 63, wherein the film is a vinyl polymer obtained by polymerization of a monomer of the following formula (I): 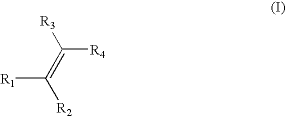
in which R₁, R₂, R₃ and R₄ are independently selected from the group consisting of hydrogen atoms, alkanes, alkenes, alkynes, amides, aldehydes, ketones, carboxylic acids, esters, acid halides, anhydrides, nitriles, amines, thiols and phosphates, ethers, homo-and heterocyclic aromatics and any cyclic group comprising these functional groups, and any group carrying several of these functional groups;
or of a mixture of different monomers of formula (I).

65. The process of claim 63, wherein the film is of polymethacrylonitrile or of poly(methylmethacrylate).

66. The process of claim 63, wherein the label is selected from the group consisting of a fluorescent label, a phosphorescent label and a chemiluminescent label.

67. The process of claim 66, comprising measuring the intensity of fluorescence emitted over the emission wavelength of the fluorescent label.
